(19) United States
(12) Patent Application Publication
(10) Pub. No.: US 2023/0270914 A1
(43) Pub. Date: Aug. 31, 2023

(54) HAEMOSTATIC MATERIAL

(71) Applicant: Medtrade Products Limited

(72) Inventors: Craig HARDY, Cheshire (GB); Guy EASON, Cheshire (GB); Andrew HOGGARTH, Cheshire (GB)

(21) Appl. No.: 18/158,156

(22) Filed: Jan. 23, 2023

Related U.S. Application Data
(63) Continuation of application No. 14/004,497, filed on Dec. 6, 2013, now abandoned.

(30) Foreign Application Priority Data
Mar. 11, 2011 (GB) 1104175.3

(51) Int. Cl. A61L 26/00; A61L 15/44; A61L 15/58; A61L 15/28; A61F 13/00

(52) U.S. Cl. CPC A61L 26/0052; A61F 13/00063; A61L 15/28; A61L 15/44; A61L 15/58; A61F 2013/00472; A61L 2300/418; A61L 2400/04

(57) ABSTRACT

The present invention relates to a haemostatic material comprising a haemostat agent and a bioadhesive agent. Such a haemostatic material is useful, for example, in effectively controlling bleeding with a reduced compression period compared to the TCCC guidance of a minimum of three minutes compression using a haemostatic bandage.

### TECHNICAL FIELD

The present invention relates to a haemostatic material for use in controlling bleeding.

### BACKGROUND

There are many circumstances in which animals, both human and non-human, may become injured or wounded causing bleeding. In the case of minor wounds, the bleeding may be stemmed by the natural haemostatic mechanisms of the body which lead to coagulation of the blood to form solid clots which prevent haemorrhage and aid repair of damaged blood vessels.

Traditionally the primary technique adopted for stemming blood flow from a wound is the application of continuous pressure to the wound. This enables clotting factors to collect at the wound site and form a congealed blood mass to stem blood flow. However, this technique is not suitable for severe wounds and wounds having multiple bleeding points. Therefore, bleeding out continues to be a major cause of death.

Death caused by bleeding out is a particular problem on the battlefield. Typically, wounds arising in this situation are accompanied by significant bleeding, and many result in death. Bleeding out is also a significant cause of death amongst the civilian population following trauma.

Attempts have been made to provide products which facilitate the stemming of blood flow from a wound. These include a product sold under the brand name Quick-clot® QUIKCLOT (Z-Medica Corp., Wallingford, CT). Simplistically, this product contains a carrier material which is coated with an active compound, which, when applied to the wound with pressure, is able to stem the blood flow. More specifically, Quick-clot® QUIKCLOT comprises a zeolite compound which absorbs water from the blood flowing from a wound, such that the clotting factors present in the blood become concentrated and the blood coagulates more quickly, thereby the zeolite and the coagulated blood together form a coagulum to stem blood flow.

Whilst effective, these compositions are not without problems as they require continuous pressure to control the bleeding. The guidance provided by the Tactical Combat Casualty Care (TCCC) in November 2009 indicated that a minimum of three minutes compression should be applied when using a haemostatic bandage, specifically Combat Gauze® QUIKCLOT COMBAT GAUZE.

### SUMMARY

Therefore, it is an object of the present invention to provide a haemostatic material which is effective at controlling the flow of blood from a wound, is easy and safe to use and requires a reduced compression time.

### DETAILED DESCRIPTION

According to a first aspect of the present invention, there is provided a haemostatic material comprising a haemostat agent and a bioadhesive agent.

By “haemostat agent” it is meant a substance that promotes haemostasis. The haemostat agent may be capable of producing a clot or plug to stop or reduce bleeding when brought into contact with blood.

A physiological target site for the haemostatic material may be any site in or on the body of an animal. The animal may be a human or a non-human animal. The physiological target site may be a wound or it may be an opening in a body caused during a medical procedure, for example during surgery. Hereinafter, the physiological target site is referred to as a wound for convenience and illustrative purposes only.

Beneficially, the haemostatic material of the present invention can be applied by a person with only basic medical training. It is a matter of simply applying the material to the physiological target site followed by pressure.

Further still, the haemostatic material is easy to handle and apply. It is typically stored dry prior to application.

Products which take advantage of biological processes tend to be temperature dependent. Often patients suffering blood loss are either very hot due to exertions on the battlefield or very cold as they have been exposed to cold conditions. Currently available products are less effective at such temperature extremes. Advantageously, the material of the present invention is substantially unaffected by temperature fluctuations and therefore works equally well at temperatures both above and below normal body temperatures. By “normal body temperature” it is meant about 37° C.

The haemostatic material of the present invention is capable of effectively controlling bleeding with a reduced compression period compared to the TCCC guidance of a minimum of three minutes compression using haemostatic bandage. Advantageously, this results in a subject being stabilised in a shorter time period before deployment to a medical area.

The haemostat agent may be any material with haemostatic properties. Examples of haemostat agents include oxidised regenerated cellulose, kaolin, gelatin, calcium ions, zeolite, collagen or chitosan. The haemostat agent is preferably a chitosan salt. Chitosan is a derivative of solid waste from shell fish processing and can be extracted from fungus culture. It is a water insoluble cationic polymeric material. Therefore, chitosan for use with the present invention is first converted into a water soluble salt. The chitosan salt is soluble in blood to fonn a gel which stems blood flow.

Chitosan salts are ideally suited for the applications described herein as chitosan is readily broken down in the body. Chitosan is converted to glucosamine by the enzyme lysozyme and is therefore excreted from the body naturally. It is not necessary to take any measures to remove the chitosan from the body.

Furthermore, chitosan salts exhibit mild antibacterial properties and as such their use reduces the risk of infection.

Exemplary chitosan salts which are suitable for use with the present invention include, but are not limited to, any of the following either alone or in combination: acetate, lactate, succinate, malate, sulphate or acrylate. They are typically in powder form.

Good results have been observed wherein the chitosan salt is chitosan succinate.

The chitosan salt is prepared by combining chitosan with an appropriate acid. It will be appreciated that the acid may be any inorganic or organic acid which yields a chitosan salt which is soluble under the conditions associated with a human or animal body, particularly in blood. Suitable acids would be recognised by a skilled person. For example, chitosan phosphate is insoluble in such conditions and so phosphoric acid is unsuitable.

The haemostat agent may constitute at least 20% by weight of the haemostatic material, or more typically at least about 80% by weight. Typically, the haemostat agent constitutes from 20-99% by weight of the haemostatic material, preferably from 45-95% by weight of the haemostatic material.

The haemostat agent is typically granular, but may comprise short fibres, sponges, fabrics, films, powders, liquid, gels or liquid coating. The short fibres may be no more than about 7.5 mm in length, more typically no more than about 5 mm in length.

The haemostat agent typically has a pH of from about 3.5 to about 8.0. The pH is largely dependent upon the particular haemostat agent used, as they each have a different pH.

By “bioadhesive agent”, it is meant a natural or synthetic biocompatible substance that binds to a biological substrate. The biological substrate may be, for example, moist tissue at a wound site. In effect, a bioadhesive agent may promote adhesion between two materials, one of which is biological in nature, such that the materials are held together for an extended period of time. The bioadhesive agent typically exhibits low adhesion to dry surfaces, for example gloves or intact skin, and high adhesion to wet/moist surfaces, for example wounds or internal organs. Consequently, the haemostatic material comprising the bioadhesive agent and the haemostat agent should preferably exhibit low adhesion to dry surfaces and high adhesion to wet/moist surfaces. Preferably, the haemostatic material exhibits no adhesion to dry surfaces. Beneficially, this property of the bioadhesive agent provides a haemostatic material that is both easy to handle and enables the haemostatic material to effectively control bleeding within a reduced compression period compared to the TCCC guidance of a minimum of three minutes compression.

The bioadhesive agent should preferably be compatible with the haemostat agent and not interfere with the efficacy of the haemostatic material. The bioadhesive agent is typically a solid, dry, material.

By ‘low adhesion’, it is meant adhesion to a surface with a peel force of 0.05 N per 25 mm of material (i.e. 0.05 N/25 mm) or below. No adhesion is effectively measured as 0.0 N/25 mm.

By ‘high adhesion’, it is meant adhesion to a surface with a peel force of 0.25 N/25 mm or above. Preferably, the adhesion to a wet/moist surface exhibits a peel force of 0.7 N/25 mm or above and more preferably 1.0 N/25 mm or above. The adhesion to a wet/moist surface typically exhibits a peel force in the range 0.6-2.0 N/25 mm

Thus, the bioadhesive agent may promote the adhesion of the haemostatic agent to moist tissue at the wound site. Beneficially, this allows the compression time required for clotting to be reduced without the blood pressure forcing the haemostatic agent from the wound site.

The bioadhesive agent may constitute up to 90% by weight of the haemostatic material. Preferably, the bioadhesive agent may constitute up to 20% by weight of the haemostatic material, more preferably from 2 to 20% by weight of the haemostatic material, even more preferably from 5 to 10% by weight of the haemostatic material and most preferably from 7 to 8% by weight of the haemostatic material. At these preferred ranges, the bioadhesive agent is optimised for adhesion to the wet or moist tissue without causing adverse effects upon removal, such as for example wound re-opening.

The bioadhesive agent should be a material which generates a high adhesion when applied to wet/moist substrates. The bioadhesive agent may be selected from any of the following either alone or in combination: carbomers, polyvinyl alcohol (PVA), polyvinylpyrrolidone (PVP), 2-acrylamido-2-methylpropane sulfonic acid, or a high molecular weight acrylic acid polymer cross-linked with divinyl glycol or the salts of polyacrylic acid cross-linked with divinyl glycol. Preferably, the bioadhesive agent comprises high molecular weight cross-linked polymers of acrylic acid. By ‘high molecular weight’ it is meant a molecular weight of at least 50000 g/mol. Preferably, the molecular weight is at least 60000 g/mol and more preferably from 100000 to 300000 g/mol. In such embodiments, the bioadhesive agent may be a homopolymer comprising a polymer of acrylic acid cross-linked with ally! sucrose or ally! pentaerythritol; a copolymer comprising a polymer of acrylic acid and C10-C30 alkyl acrylate cross-linked with allyl pentaerythritol; a carbomer homopolymer or copolymer comprising a block copolymer of polyethylene glycol and a long chain alkyl acid ester; or mixtures thereof.

For example, the bioadhesive agent may be selected from any of the following, either alone or in combination: Carbopol® NF934, NF974, NF971 and NF980.

The bioadhesive agent provides the composition of the present invention with excellent wet stick properties. By “wet stick” it is meant adhesion to wet or moist tissue. This allows for the bioadhesive agent to promote adhesion between the haemostat agent and moist tissue at the wound site.

In some embodiments, the haemostat agent and the bioadhesive agent are typically present in a ratio of at least 3:1. Typically, the haemostat agent and bioadhesive agent are present in a ratio of at least 4:1 and more preferably in a ratio of at least 9:1.

The haemostatic material of the present invention should comprise a sufficient amount of bioadhesive agent to effectively control bleeding within a reduced compression period compared to the TCCC guidance of a minimum of three minutes compression. However, in some embodiments, high proportions of the bioadhesive material may not result in the improved characteristics. Therefore, the present invention is ideally carried out using the ranges described herein.

The haemostatic material of the present invention may comprise an anionic bioadhesive agent in combination with a cationic haemostat agent. In such embodiments, at least a portion of the anionic bioadhesive agent may react with the cationic haemostat agent. The reaction between the bioadhesive agent and the haemostat agent may occur to a varying degree. For example, all of the anionic bioadhesive agent may not react with the cationic haemostat agent, such that the resulting haemostatic material comprises a mixture of unreacted haemostat agent, unreacted bioadhesive agent and/or reacted bioadhesive/haemostat, or all of the anionic bioadhesive agent may react with the cationic haemostat agent.

The haemostat agent may further comprise an inert material. By “inert” it is meant a material having non-haemostatic or poorly haemostatic properties and having low adhesion to wet/moist surfaces.

Exemplary inert materials include but are not limited to non-haemostatic cellulose, non-haemostatic sand, non-haemostatic clay, non-haemostatic alginate, microcrystalline cellulose, guar gum, xanthan gum, non-haemostatic chitosan, non-haemostatic chitin, dextran, sucrose, lactose, pectin, carboxymethylcellulose, hydroethyl cellulose, ground corn meal, polyacrylic acid, barium sulphate, starch, or combinations of any two or more thereof. Typically, one or more inert materials selected from non-haemostatic chitosan, non-haemostatic chitin and carboxymethylcellulose are used.

The inert material may be added to the haemostat agent in an amount up to about 95% by weight of the total composition, typically up to about 80% by weight, and more typically up to about 50% by weight. The inert material is typically blended with the haemostat agent, but may be dispersed in solution with the haemostat agent and dried.

Typically, the inert material is granular, but can be in the form of a powder, foam, fibres, or films.

The haemostat agent may further comprise a medical surfactant. By “medical surfactant” it is meant any surfactant that is pharmaceutically acceptable for contact with or administration to a human or animal body and does not cause any significant detrimental effects to the human or animal body. Exemplary medical surfactants for use in the present invention include any of the following either alone or in combination: block copolymers based on ethylene oxide and propylene oxide (e.g. BASF Pluronics®), glycerol, polyethylene glycol, propylene glycol, fatty acids such as lauric acid, oleic acid, other fatty acids and fatty acid salts, silicone based surfactants and emulsifiers. Typically, the medical surfactants include lauric acid and oleic acid.

The medical surfactant may typically constitute from about 0.001 to about 10% by weight of the haemostat agent.

More advantageously, the medical surfactant constitutes from about 0.5 to about 1% by weight of the haemostat agent. Advantageously, the presence of a surfactant gives rise to excellent wetting out properties. The way in which the haemostat agent wets out is important to its performance. That is, the haemostat agent can absorb the blood too quickly and simply mix with the blood without sufficient gelation having occurred to form a gel clot which is capable of stemming blood flow. On the other hand, if the haemostat agent absorbs the blood too slowly gelation occurs in only a small amount of the haemostat agent, generally the first few millimetres depth of the haemostat agent closest to the wound site. In this case the gel clot which forms is not sufficiently dense to stem the blood flow for a sufficient period of time to allow the patient to be moved to a medical centre. Typically, such a gel clot will break up as the patient is moved and bleeding will resume.

It has been found that by adding an amount of an inert material and/or an amount of a medical surfactant to the haemostat agent, i.e. in effect diluting the quantity of haemostat agent, the performance of the haemostat agent is actually enhanced further. A combination of the inert material and the medical surfactant together is particularly advantageous as the presence of the inert material further enhances the properties of the medical surfactant, and vice versa.

The particle size of the haemostat agent can affect the performance of the haemostatic material of the present invention. The particle size is measured by the size of sieve through which the paiticle will pass or be retained by.

For example, when the haemostat agent is in particulate or granular form, it may have an average particle size of greater than about 200 mesh such that it will not pass through a 200 mesh sieve. The average particle size may typically be greater than about 100 mesh, still more typically greater than about 50 mesh, and it is not desired that the particles or granules are able to pass through a 40 mesh sieve.

More advantageously, the particle size of the inert material will be substantially equivalent to that of the haemostat agent. By “substantially equivalent” it is meant that the relative sizes of the particles do not differ by more than about 25%, more typically by more than about 10%. The optimum particle size is achieved by grinding the haemostat agent and sorting by any suitable means such as sieving. Such sizing processes are well known to those skilled in the art and will not be described further.

The haemostatic material may be administered to the wound in any particular form, such as for example, a dry powder, solution, foam or gel.

The haemostatic material may be applied to a carrier material for application to the wound site. The carrier material may comprise a viscose non-woven material, or alternatively it may comprise a thin flexible substrate, a woven gauze, a film, a foam, or a sheet gel. The material may or may not be degradable in conditions associated with wounds in or on a human or animal body. In one example, the material of the carrier material may be safely degradable in the body so that the whole haemostatic material piece can be left in place after surgical use or treatment. Examples of safe and degradable materials include, but are not limited to, oxidised cellulose, gelatin, dextran, collagen, polycaprylactone, polylactide acid, polylactide-co-glycolide, polyglycolide, chitin, etc.

The haemostat agent may be applied to the carrier material by a variety of methods. These include bonding the haemostat agent to the carrier material using an adhesive; applying a solution containing the haemostat agent to the carrier material, coating the carrier material and drying the solution; or by heat bonding. The haemostat agent may also be incorporated into the carrier material during the processing of the carrier materials.

The material may take any suitable form and may be provided in a range of different sizes, shapes and thicknesses necessary to deal with a wound, such as square, rectangular, circular or elliptical. For example, the material may be a generally flat shape with little height relative to its width/depth. Any regular or irregular shape may be employed. It may be provided in large sheets which can be cut to the required size.

When a chitosan salt is used as the haemostat agent in the material of the invention, an active base is prepared by preparing a mixture of chitosan in particulate, granular, powder, flake or short fibrous form and an appropriate acid in a solvent in which the chitosan is insoluble (typically 80:20 ethanol:water). The solvent is evaporated to provide a substantially active base material. The active base material may then be combined with an inert material and/or a medical surfactant as desired to provide the haemostat agent.

The haemostatic material may be provided in a sterile or non-sterile form. Where the material is provided in a sterile form, sterilisation may be carried out using any of the conventionally known methods, such as gamma irradiation, electron beam treatment, heat treatment, ethylene oxide (EtO) sterilization etc. A material in a non-sterile form may be provided in combination with one or more preservatives or antimicrobial agent, such as silver and its salts.

According to a second aspect of the present invention, there is provided a method of haemostasis, the method comprising the steps of applying the haemostatic material as described herein to a physiological target site; and applying pressure to the haemostatic materiaL

According to a third aspect of the present invention, there is provided a haemostatic material as described herein for use in stemming blood flow from a physiological target site.

The pressure may be applied to the target site for at least one minute. In some embodiments, the pressure may be applied to the wound site for at least two minutes. An advantage of the present invention is the relatively quick time taken to sufficiently clot blood flowing from a wound site. Thus, sufficient clotting forms within three minutes such that the pressure may be applied to the target site for a shorter time to obtain the desired effect. In some embodiments, the pressure may be applied to the wound site for less than two minutes to have the desired effect, and preferably less than one minute.

According to a fourth aspect of the present invention, there is provided a carrier material comprising a haemostatic material as described herein applied to the carrier material.

The carrier material may comprise any of the features of the carrier material described hereinbefore. Preferably, the carrier material comprises a viscose gauze.

According to a fifth aspect of the present invention, there is provided a method of manufacturing a haemostatic material, the method comprising the step of combining a haemostat agent with a bioadhesive agent.

Preferably, the method of manufacturing the haemostatic material comprises the steps of (1) dispensing a pre-determined weight of a haemostat agent and optionally an inert material into a mixing vessel; (2) dispensing a pre-determined weight of a bioadhesive agent into the mixing vessel containing the haemostat and optional inert material; and (3) mixing the haemostat agent and bioadhesive agent.

The haemostat agent may be dispersed into a solution with the inert material and bioadhesive and mixed. The solution may subsequently be evaporated.

Embodiments of the present invention will now be described further in the following non-limiting examples with reference to the accompanying drawings in which:

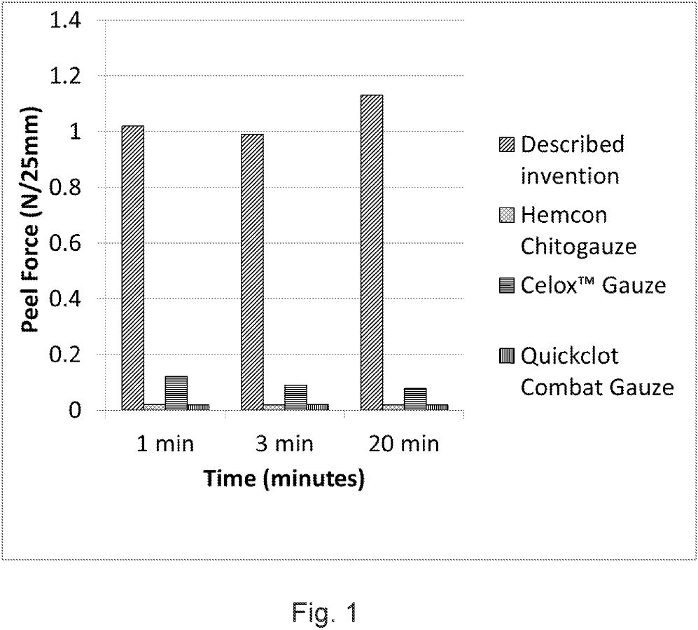

FIG. 1 is a graph displaying the peel force required to remove a haemostatic material from a wound versus time for compositions of the present invention and known devices;

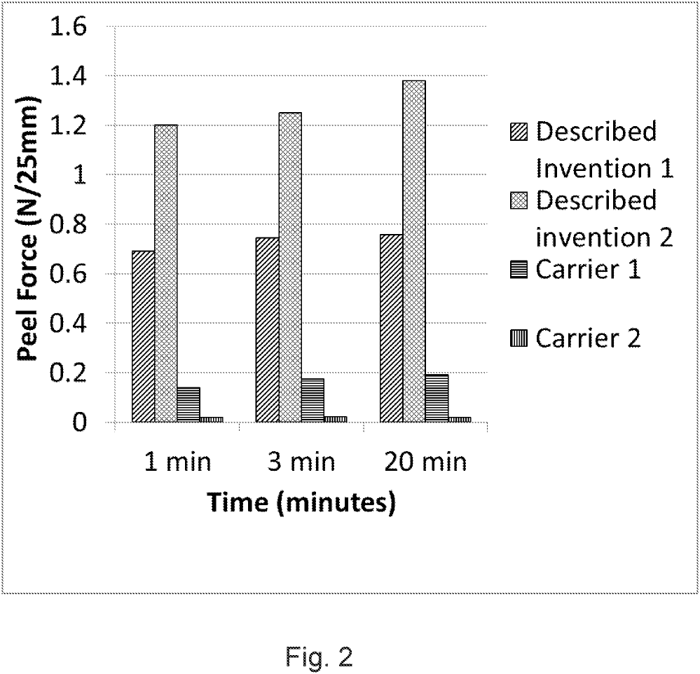

FIG. 2 is a graph displaying the peel force required to remove a haemostatic material from a wound versus time for compositions of the present invention and comparative examples.

### EXAMPLE 1

A 5 wt% bioadhesive agent (high molecular weight cross-linked polymers of acrylic acid (Carbopol® NF934)) was blended with a chitosan lactate/non-haemostatic chitosan blend. The mixture was double-coated onto viscose gauze at a coat weight of 40 gsm. This provided a haemostatic material referred to herein as ‘Described Invention’

### EXAMPLE 2

A 10 wt% bioadhesive agent (high molecular weight cross-linked polymers of acrylic acid (Carbopol® NF934)) was blended with a chitosan lactate/non-haemostatic chitosan blend. The mixture was double-coated onto viscose gauze at a coat weight of 40 gsm.

The effectiveness of the haemostatic materials of Examples 1 and 2 was assessed in vivo and in vitro as described below.

### In Vitro

An in vitro adhesion model was used to assess the ability of the haemostatic material to adhere to moist tissue. The model incorporated using the underside of pork belly. The pork belly was kept in cool conditions for 24 hours prior to testing (3° C.) to ensure all moisture was retained within the pork belly. Sample strips of the test articles were cut to 25 mm width. The test articles were applied to the pork belly and a 5 kg weight applied over the top. The adhesion to the moist pork belly was assessed at 1 min, 3 min and 20 min using a tensiometer.

The results presented in FIG. 1 show that the haemostatic material of the present invention has a significantly greater adhesion compared to the known devices. The dressings tested were Hemeon Chitogauze®, Celox® gauze and Quickclot® Combat Gauze®.

For a comparative analysis of the enhanced effect of haemostatic material of the present invention versus haemostat agents alone, further work was undertaken using oxidised regenerated cellulose with and without high molecular weight cross-linked polymers of acrylic acid and a gauze impregnated with kaolin with and without high molecular weight cross-linked polymers of acrylic acid.

Oxidised regenerated cellulose comprising 5 wt% of a high molecular weight cross-linked polymer of acrylic acid (Carbopol® NF980) is shown as Described Invention 1. A gauze impregnated with kaolin comprising 20 wt% of a high molecular weight cross-linked polymer of acrylic acid (Carbopol® NF980) is shown as Described Invention 2. Carrier 1 and Carrier 2 comprise oxidised regenerated cellulose and gauze impregnated with kaolin respectively. The results are shown in FIG. 2. As can be seen, the haemostatic materials of the present invention have a significantly greater adhesion to the tissue compared to the oxidised regenerated cellulose and gauze impregnated with kaolin.

### In Vivo

To confirm the wet adhesion gave real advantages in compression time and provided evidence of efficacy with 1 minute compression the compositions of Examples 1 and 2 were tested in a porcine model using a 6 mm femoral artery sever model. A 6 mm sever was surgically made to the femoral artery of a porcine model. The artery was allowed to bleed out for a period of 45 seconds following which the haemostatic material was applied to the bleed site and pressure applied for a period of one minute. Following the compression period the wound was assessed for bleeding. If bleeding re-occurred, the haemostatic material was removed and a new sample re-applied to the bleed site followed by one minute pressure. Any re-bleeding after this point was classified as a fail.

The results demonstrated that 94% of the models treated obtained haemostasis within the protocol in the femoral artery model.

### Adhesion

Further testing was conducted to assess the adhesion of the haemostatic material of the present invention to a dry tissue surface, compared to the above referenced tests for adhesion to a wet tissue surface. The haemostatic materials of Described Invention, Described Invention 1 and Described Invention 2 were tested in comparison to prior art devices including Hemeon Chitogauze®, Celox® gauze, Quickclot Combat Gauze® and oxidised regenerated cellulose. The peel force of each material was assessed on dry and wet tissue after time intervals of 1, 3 and 20 minutes. The results are shown in Table 1 below.

TABLE I

| | | Peel force (N/25mm) | | |
|---|---|---|---|---|
| | Tissue Type | 1 min | 3 min | 20 min |
| Hemeon Chitogauze® | Wet | 0.021 | 0.018 | 0.019 |
| Celox® Gauze | Wet | 0.12 | 0.09 | 0.078 |
| Quickclot® Combat Gauze® | Wet | 0.019 | 0.021 | 0.019 |
| Oxidised Regenerated cellulose | Wet | 0.14 | 0.174 | 0.192 |
| Described Invention | Wet | 1.01 | 0.98 | 1.15 |
| Described Invention 1 | Wet | 0.692 | 0.745 | 0.758 |
| Described invention 2 | Wet | 1.2 | 1.25 | 1.38 |
| Hemeon Chitogauze® | Dry | No adhesion | No adhesion | No adhesion |
| Celox® gauze | Dry | No adhesion | No adhesion | No adhesion |
| Quickclot® Combat Gauze® | Dry | No adhesion | No adhesion | No adhesion |
| Oxidised Regenerated cellulose | Dry | No adhesion | No adhesion | No adhesion |
| Described Invention | Dry | No adhesion | No adhesion | No adhesion |
| Described Invention 1 | Dry | No adhesion | No adhesion | No adhesion |
| Described Invention 2 | Dry | No adhesion | No adhesion | No adhesion |

As can be seen in Table 1, the haemostatic materials of the present invention exhibited no adhesion to dry tissue and high adhesion to wet tissue at each of the three time intervals. The results show a higher wet adhesion of the haemostatic materials of the present invention as compared to the known devices.

It is of course to be understood that the present invention is not intended to be restricted to the foregoing examples which are described by way of example only.

1. A haemostatic material comprising a haemostat agent and a bioadhesive agent.

2. A haemostatic material as claimed in claim 1, wherein the haemostat agent is selected from the list consisting of: oxidised regenerated cellulose, kaolin, gelatin, calcium ions, zeolite, collagen, chitosan and chitosan derivatives.

3. A haemostatic material as claimed in claim 1, wherein the haemostat agent is a chitosan salt.

4. A haemostatic material as claimed in claim 3, wherein the chitosan salt comprises one or more chitosan salts selected from: chitosan acetate, chitosan lactate, chitosan succinate, chitosan malate, chitosan sulphate and chitosan acrylate.

5. A haemostatic material as claimed in claim 4, wherein the chitosan salt is chitosan succinate.

6. A haemostatic material as claimed in claim 1, wherein haemostat agent constitutes at least 20% by weight of the haemostatic material.

7. A haemostatic material as claimed in claim 6, wherein the haemostat agent constitutes at least 80% by weight of the haemostatic material.

8. A haemostatic material as claimed in claim 5, wherein the haemostat agent constitutes from 20 to 99% by weight of the haemostatic material.

9. A haemostatic material as claimed in claim 8, wherein the haemostat agent constitutes from 45 to 95% by weight of the haemostatic material.

10. A haemostatic material as claimed in claim 1, wherein the haemostat agent is granular, short fibres, sponges, fabrics, films, powders, liquid, gels or liquid coating.

11. A haemostatic material as claimed in claim 10, wherein the short fibres are no more than 7.5 mm in length.

12. A haemostatic material as claimed in claim 11, wherein the short fibres are no more than 5 mm in length.

13. A haemostatic material as claimed in claim 1, wherein the haemostat agent has a pH of from about 3.5 to about 8.0.

14. A haemostatic material as claimed in claim 1, wherein the adhesion of the material to dry surfaces has a peel force of 0.05 N per 25 mm or below and the adhesion to wet/moist surfaces has a peel force of 0.25 N per 25 mm or above.

15. A haemostatic material as claimed in claim 14, wherein the adhesion to a wet/moist surface has a peel force of 0.7 N per 25 mm or above.

16. A haemostatic material as claimed in claim 15, wherein the adhesion to a wet/moist surface has a peel force of 1.0 N per 25 mm or above.

17. A haemostatic material as claimed in claim 1, wherein the adhesion of the material to wet/moist surfaces has a peel force in the range 0.6 to 2.0 N per 25 mm.

18. A haemostatic material as claimed in claim 1, wherein the bioadhesive agent constitutes up to 90% by weight of the haemostatic material.

19. A haemostatic material as claimed in claim 18, wherein the bioadhesive agent constitutes up to 20% by weight of the haemostatic material.

20. A haemostatic material as claimed in claim 19, wherein the bioadhesive agent constitutes from 2 to 20% by weight of the haemostatic material.

21. A haemostatic material as claimed in claim 20, wherein the bioadhesive agent constitutes from 5 to 10% by weight of the haemostatic material.

22. A haemostatic material as claimed in claim 21, wherein the bioadhesvie agent constitutes from 7 to 8% by weight of the haemostatic material.

23. A haemostatic material as claimed in claim 1, wherein the bioadhesive agent is selected from the following, either alone or in combination: carbomers, polyvinyl alcohol, polyvinylpyrrolidone, 2-acrylamido-2-methylpropane sulfonic acid, an acrylic acid polymer having a molecular weight of at least 50000 g/mol cross-linked with divinyl glycol or the salts of polyacrylic acid cross-linked with divinyl glycol.

24. A haemostatic material as claimed in claim 23, wherein the bioadhesive agent comprises a homopolymer comprising a polymer of acylic acid cross-linked with allyl sucrose or allyl pentaerythritol; a copolymer comprising a polymer of acrylic acid and C10-C30 alkyl acrylate cross linked with allyl pentaerythritol; a carbomer homopolymer or copolymer comprising a block copolymer of polyethylene glycol and a long chain alkyl acid ester, or mixtures thereof.

25. A haemostatic material as claimed in claim 1, wherein the haemostat agent and the bioadhesive agent are present in a ratio of at least 3:1.

26. A haemostatic material as claimed in claim 25, wherein the haemostat agent and the bioadhesive agent are present in a ratio of at least 4:1.

27. A haemostatic material as claimed in claim 26, wherein the haemostat agent and the bioadhesive agent are present in a ratio of at least 9:1.

28. A haemostatic material as claimed in claim 1, wherein the bioadhesive agent is anionic and the haemostat agent is cationic.

29. A haemostatic material as claimed in claim 1, further comprising an inert material.

30. A haemostatic material as claimed in claim 29, wherein the inert material comprises one or more components selected from: non-haemostatic cellulose, non-haemostatic sand, non-haemostatic clay, non-haemostatic alginate, microcrystalline cellulose, guar gum, xanthan gum, non-haemostatic chitosan, non-haemostatic chitin, dextran, sucrose, lactose, pectin, carboxymethylcellulose, hydroethyl cellulose, ground corn meal, polyacrylic acid, barium sulphate, starch, or combinations of any two or more thereof.

31. A haemostatic material as claimed in claim 29, wherein the inert material constitutes up to about 95% by weight of the haemostatic material.

32. A haemostatic material as claimed in claim 29, wherein the inert material is in the form of granules, a powder, foam, fibres or a film.

33. A haemostatic material as claimed in claim 1, further comprising a medical surfactant.

34. A haemostatic material as claimed in claim 33, wherein the medical surfactant comprises one or more components selected from: block copolymers based on ethylene oxide and propylene oxide, glycerol, polyethylene glycol, propylene glycol, fatty acids, fatty acid salts, silicone based surfactants and emulsifiers.

35. A haemostatic material as claimed in claim 33, wherein the medical surfactant is a fatty acid selected from lauric acid and oleic acid.

36. A haemostatic material as claimed in claim 33, wherein the medical surfactant constitutes from about 0.001 to about 10% by weight of the haemostat agent.

37. A haemostatic material as claimed in claim 1, wherein the haemostat agent comprises particles that will not pass through a 200 mesh sieve.

38. A haemostatic material as claimed in claim 29, wherein the particle size of the inert material will be substantially equivalent to that of the haemostat agent.

39. A haemostatic material as claimed in claim 1 in dry powder, solution, foam or gel form.

40. A carrier material comprising a haemostatic material as claimed in claim 1 applied to the carrier material.

41. A carrier material as claimed in claim 40 in the form of a viscose non-woven material, a thin flexible substrate, a woven gauze, a film, a foam, or a sheet gel.

42. A carrier material as claimed in claim 40, wherein the carrier material is made from oxidised cellulose, gelatin, dextran, collagen, polycaprylactone, polylactide acid, polylactide-co-glycolide, polyglycolide, or chitin.

43. A method of haemostasis, the method comprising the steps of applying the haemostatic material as claimed in claim 1 to a physiological target site; and applying pressure to the haemostatic material.

44. A method as claimed in claim 43, wherein the pressure is applied for less than three minutes.

45. A haemostatic material as claimed in claim 1 for use in stemming blood flow from a physiological target site.

46. A method of manufacturing a haemostatic material, the method comprising the step of combining a haemostat agent with a bioadhesive agent.

47. A method as claimed in claim 44, wherein the method comprises the steps of:
1. dispensing a pre-determined weight of a haemostat agent and optionally an inert material into a mixing vessel;
2. dispensing a pre-determined weight of a bioadhesive agent into the mixing vessel containing the haemostat and optional inert material; and
3. mixing the haemostat agent and bioadhesive agent.

48. Use of a haemostatic material as claimed in claim 1 in reducing or stopping blood flow from a physiological target site.

49. (canceled)

50. A haemostatic material as claimed in claim 1, wherein the haemostat agent is selected from the list consisting of: oxidised regenerated cellulose, kaolin, gelatin, calcium ions, zeolite, collagen, chitosan and chitosan derivatives, and wherein the bioadhesive agent is present in an amount of from 2-20% by weight of the haemostatic material.
